(19) United States
(12) Patent Application Publication
(10) Pub. No.: US 2009/0111947 A1
(43) Pub. Date: Apr. 30, 2009

(54) COMPOUNDING AGENT FOR RUBBER VULCANIZATION CONTAINING AMINO ALCOHOL SALT COMPOUND OF AROMATIC CARBOXYLIC ACID, PRODUCTION METHOD THEREOF AND RUBBER COMPOSITION CONTAINING THE SAME

(75) Inventors: Wonmun Choi, Hiratsuka-Shi (JP); Takashi Matsuda, Hiratsuka-Shi (JP)

Correspondence Address: CONNOLLY BOVE LODGE & HUTZ LLP, 1875 EYE STREET, N.W., SUITE 1100, WASHINGTON, DC, 20006, US

(73) Assignee: The Yokohama Rubber Co., Ltd., Tokyo (JP)

(21) Appl. No.: 12/256,563

(22) Filed: Oct. 23, 2008

(30) Foreign Application Priority Data
Oct. 25, 2007 (JP) 2007-277982

(51) Int. Cl. C08F 8/32; C07C 63/08; C08C 19/22; C07C 63/28

(52) U.S. Cl. 525/380; 562/493

(57) ABSTRACT

A compounding agent for rubber vulcanization comprising an amino alcohol salt compound of an aromatic carboxylic acid having the formula (I) obtained by reacting an aromatic carboxylic acid and amino alcohol:

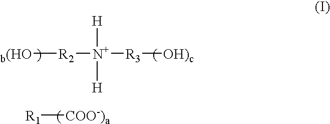

wherein R₁ is a C₆ to C₂₄ aromatic hydrocarbon which may have a hydroxy group and/or a substituent group, R₂ and R₃ are independently hydrogen or a C₁ to C₂₀ organic groups that may have a hetero atom and/or a substituent group, and R₂ and R₃ may bond with each other to form a ring, a is 1 or 2, b and c are 0 or 1 and b+c is 1 to 2.

### TECHNICAL FIELD

The present invention relates to a novel compounding agent for rubber vulcanization containing an amino alcohol salt of an aromatic carboxylic acid, the production method thereof and a rubber composition containing the same.

### BACKGROUND ART

Generally, as rubber vulcanization accelerators, thiurams, sulfenamides, mercaptobenzothiazoles and the like are used. Sulfenamides are delayed accelerators wherein N—S bonds dissociate by the heat during vulcanization and produce mercaptobenzothiazole and amine. It is known that the resultant mercaptobenzothiazole acts as a vulcanization accelerator and the amine is positioned at the zinc white and plays an important role in accelerating the vulcanization reaction by activation of the vulcanization system and reaction with the vulcanization intermediate (see Chapman, A. V., Porter, M.: “Sulphur Vulcanization Chemistry” in the Natural Rubber Science and Technology, Roberts, A. D. ed., Oxford Science Publications, London (1988)).

### DISCLOSURE OF THE INVENTION

The joint use of amines may be considered for the purpose of improving the vulcanization acceleration capability, but in this case, the reactivity of the amines is high, and, therefore, there is the problem that they react with the sulfur or other vulcanization agents even at a low temperature to thereby provide a detrimental effect on the scorching and other aspects of the processability.

Accordingly, an object of the present invention is to provide a compounding agent for rubber vulcanization comprising an amino salt compound of an aromatic carboxylic acid capable of improving the vulcanization speed (or rate) and vulcanized rubber physical properties, as well as to provide a production method thereof and a rubber composition containing the same.

In accordance with the present invention, there is provided a compounding agent for rubber vulcanization comprising an amino alcohol salt compound of an aromatic carboxylic acid having the formula (I) obtained by reacting an aromatic carboxylic acid with an amino alcohol:

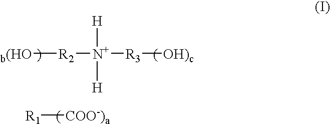

wherein R₁ is a C₆ to C₂₄ aromatic hydrocarbon which may have a hydroxy group and/or a substituent group, R₂ and R₃ are independently hydrogen or a C₁ to C₂₀ organic group which may have a hetero atom and/or a substituent group and R₂ and R₃ may bond with each other to form a ring, a is 1 or 2, b and c are 0 or 1 and b+c is 1 to 2.

In accordance with the present invention, there is further provided a method for producing a compounding agent for rubber vulcanization comprising an amino alcohol salt compound of an aromatic carboxylic acid having the formula (I) by reacting an aromatic carboxylic acid having the formula (II) and an amino alcohol having the formula (III) (see following reaction formula (1)).

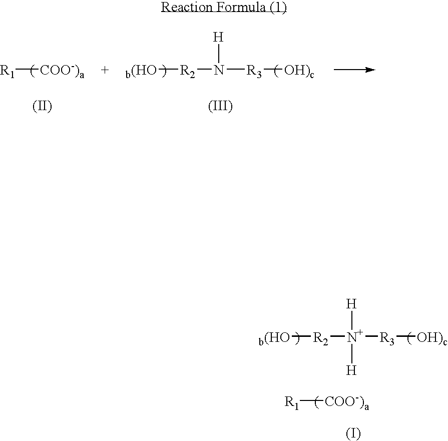

wherein R₁ is a C₆ to C₂₄ aromatic hydrocarbon which may have a hydroxy group and/or a substituent group, R₂ and R₃ are independently hydrogen or a C₁ to C₂₀ organic group which may have a hetero atom and/or a substituent group and R₂ and R₃ may bond with each other to form a ring, a is 1 or 2, b and c are 0 or 1 and b+c is 1 to 2.

According to the present invention, by using an amino alcohol salt compound of an aromatic carboxylic acid having the formula (I), a high vulcanization acceleration effect with respect to rubber blends containing a natural rubber-based rubber can be obtained and further it is possible to improve the vulcanization speed and vulcanized rubber physical properties (e.g., heat aging resistance and heat buildup property).

In this specification and in the claims which follow, the singular forms “a”, “an” and “the” include plural referents unless the context clearly dictates otherwise.

### BEST MODE FOR CARRYING OUT THE INVENTION

In formula (I), R₁ is independently a C₆ to C₂₄ aromatic hydrocarbon having a hydroxy group and/or a substituent group. As the substituent group, for example, linear hydrocarbon groups such as a methyl group, ethyl group, propyl group, butyl group, hexyl group, stearyl group, halogen groups such as an iodo, bromo, chloro, fluoro an alkoxy group, a carbonyl group, an ester group, etc. may be mentioned.

In formula (I), as the organic groups R₂ and R₃, hydrogen or, for example, linear hydrocarbon groups such as a methyl group, ethyl group, propyl group, butyl group, hexyl group, stearyl group, methylene group, ethylene group, propylene group, hexylene group; cyclic hydrocarbon groups such as a cyclobutyl group, cyclohexyl group, cyclobutylene group, cyclohexylene group, aromatic hydrocarbon groups such as a benzyl group, ethylbenzene group, xylylene group, or other alkyl aromatic group, phenylene group, naphthalene group, may be mentioned. These organic group chains may have hetero atoms such as a nitrogen atom, oxygen atom, sulfur atom. R₂ and R₃, together with their bonded nitrogen atoms, may form a piperidine group, morpholine group, thiamorpholine group, piperazine etc. When R₂ and R₃ form a heterocyclic group, together with their bonded nitrogen atoms, they may further have a substituent group on the heterocyclic ring. As examples of the substituent groups, for example, alkyl groups such as a methyl, ethyl; a halogen group such as a iodo, bromo, chloro, fluoro; an alkoxy group, a carbonyl group, an ester group, etc. may be mentioned.

As the amino alcohol having the formula (III), ethanol amine, 3-amino-1-propanol, 2-amino-1-propanol, 1-amino-2-propanol, 4-amino-1-butanol, 2-amino-1-butanol, 2-amino-2-methyl-1-propanol, 5-amino-1-pentanol, 2-amino-1-pentanol, 6-amino-2-methyl-2-heptanol, 1-amino-1-cycloheptane methanol, 2-aminocyclohexanol, 4-aminocyclohexanol, 1-aminomethyl-1-cyclohexanol, 2-(2-aminoethoxy)ethanol, 2-(methylamino)ethanol, 2-(ethylamino)ethanol, 2-(propylamino)ethanol, diethanol amine, diisopropanolamine, serinol, 2-amino-2-ethyl-1,3-propanol, 2-amino-2-methyl-1,3-propanol, 3-pyrrolidinol, 2-piperidine methanol, 2-piperidine ethanol, 3-hydroxypiperidine, 4-hydroxypiperidine, 4-aminophenetyl alcohol, 2-amino-m-cresol, 2-amino-o-cresol, 2-amino-p-cresol, 5-amino-2-methoxyphenol, 2-amino-4-chlorophenol, 4-amino-3-chlorophenol, 4-amino-2,5-dimethylphenol, tyramine, 2-amino-4-phenylphenol, 1-amino-2-napthanol, 4-amino-1-napthanol, 5-amino-1-napthanol and dopamine may be mentioned. Among these, 2-aminoethanol, 1-amino-2-propanol, 4-hydroxypiperidine, diisopropanolamine and diethanol amine are preferable due to their good industrial availability.

The amino alcohol salt compound of an aromatic carboxylic acid (I) according to the present invention can be produced, as shown in the reaction formula (1), by reacting the aromatic carboxylic acid having the formula (II) and the amino alcohol having the formula (III), wherein R is as defined above. This reaction can be carried out by mixing and reacting the compounds having the formula (II) and formula (III) in a suitable solvent (e.g., methanol, ethanol, propanol, or another aliphatic alcohol, diethyl ether, tetrahydrofuran and other ethers, acetone, 2-butanone and other ketones, etc.) In the reaction formula (1), the amino alcohol (III) is preferably reacted with the carboxylic acid group of the aromatic carboxylic acid (II) stoichiometrically, for example, in 0.9 to 1.15 equivalents.

In the reaction formula (1), as specific examples of an aromatic carboxylic acid compound (II) usable as the starting material, for example, benzoic acid, o-toluic acid, m-toluic acid, p-toluic acid, 2-fluorobenzoic acid, 2-chlorobenzoic acid, 2-bromobenzoic acid, 2-iodobenzoic acid, 3-fluorobenzoic acid, 3-chlorobenzoic acid, 3-bromobenzoic acid, 3-iodobenzoic acid, 4-fluorobenzoic acid, 4-chlorobenzoic acid, 4-bromobenzoic acid, 4-iodobenzoic acid, salicylic acid, 3-hydroxybenzoic acid, 4-hydroxybenzoic acid, o-anisic acid, m-anisic acid, p-anisic acid, 4-ethylbenzoic acid, 4-propyl benzoic acid, 4-isopropyl benzoic acid, 4-butylbenzoic acid, 4-tert-butylbenzoic acid, 4-vinylbenzoic acid, 4-ethoxybenzoic acid, 4-propoxybenzoic acid, 3,4-dimethylbenzoic acid, 2,5-dimethylbenzoic acid, 3,4-dimethoxybenzoic acid, 2,5-methoxybenzoic acid, phthalic acid, isophthalic acid, terephthalic acid, etc. may be used.

The reaction temperature of the above reaction is not particularly limited, but is preferably 0° C. to 100° C. in range. If the temperature is less than 0° C., the reaction time becomes slow, while at a temperature more than 100° C., a secondary reaction unpreferable to the products is liable to occur. The above reaction temperature is more preferably within the range of 20° C. to 70° C.

As specific examples of the vulcanization agent, usable in the present compounding agent for rubber vulcanization, for example, sulfur, organic peroxide, quinone dioxime, a metal oxide, an alkylphenol-formaldehyde resin, etc. may be mentioned.

As the compounding agent for rubber vulcanization usable jointly with the amino alcohol salt of the aromatic carboxylic acid according to the present invention, a sulfenamide-based or thiuram-based vulcanization accelerator is preferably included. By using a sulfenamide-based or thiuram-based vulcanization accelerator, it is possible to further accelerate the vulcanization of the rubber ingredient and, further, to further improve the physical properties of the obtained vulcanized rubber. As the sulfenamide-based vulcanization accelerator, for example, N-cyclohexyl-2-benzothiazolyl sulfenamide, N-t-butyl-2-benzothiazolyl sulfenamide, N-oxydiethylene-2-benzothiazolyl sulfenamide and N,N′-dicyclohexyl-2-benzothiazolyl sulfenamide may be mentioned. As the thiuram-based vulcanization accelerator, for example, tetrakis(2-ethylhexyl)thiuram disulfide, tetramethyl thiuram disulfide, tetraethyl thiuram disulfide, tetramethyl thiuram monosulfide, tetrabenzyl thiuram disulfide and dipentamethylene thiuram tetrasulfide may be mentioned.

The rubber composition of the present invention comprises an unvulcanized rubber component selected from the group consisting of diene-based rubbers and an amino alcohol salt of an aromatic carboxylic acid (I) according to the present invention. The unvulcanized rubber components which the present rubber composition may include, those selected from the group consisting of diene-based rubbers. As specific examples of diene-based rubbers, for example, natural rubber, butadiene rubber, isoprene rubber, chloroprene rubber, styrene-butadiene copolymer rubber, ethylene-propylene diene copolymer rubber and acrylonitrile-butadiene copolymer rubber may be mentioned.

In the rubber composition according to the present invention, the amino alcohol salt of an aromatic carboxylic acid (I) according to the present invention may be used, as a compounding agent for rubber vulcanization alone or together with a vulcanization agent or vulcanization accelerator generally used as a vulcanization agent or vulcanization accelerator for unvulcanized rubber in the art. The amino alcohol salt of an aromatic carboxylic acid (I) of the present invention can be used in any ratio to the total amount of the other vulcanization agents and/or vulcanization accelerators contained in the compounding agent for rubber vulcanization so long as the vulcanization and/or vulcanization acceleration action of the amino alcohol salt of the aromatic carboxylic acid is not adversely affected and an improvement of the desired vulcanization and/or a vulcanization acceleration effect and heat aging resistance can be achieved. However, to achieve the desirable vulcanization and/or vulcanization acceleration effect, the amount is preferably 0.1 to 10 parts by mass based upon to 100 parts by mass of unvulcanized rubber component selected from the group consisting of diene-based rubbers. If the amount of the amino alcohol salt of an aromatic carboxylic acid (I) is in this range, a more advantageous effects such as the ability to obtain a practical strength and rubber elasticity can be obtained. Further, the vulcanization temperature is usually preferably 140° C. to 200° C.

The rubber composition of the present invention may contain, in addition to the vulcanization accelerator, various types of agents and additives usually compounded into rubber compositions such as carbon black, silica, or other reinforcing agents, vulcanization or cross-linking agents, vulcanization or cross-linking accelerators, stearic acid, zinc oxide, magnesium oxide, and other vulcanization acceleration aids, various types of oils, an antioxidant, filler, paraffin oil or other softening agent, a plasticizer, antioxidant, etc. in the amounts generally used according to the various types of applications by general blending methods. This compounding may be obtained by kneading by a generally uses rubber kneader, for example, rolls, a Banbury mixer, a kneader, etc.

### EXAMPLES

The present invention will now be explained in further detail with reference to the Examples and Comparative Examples, but the technical scope of the present invention is by no means limited to these Examples.

### Synthesis of Compound 1

Into 300 g of toluene, 122.12 g (1 mole) of benzoic acid and 61.08 g (1 mole) of 2-aminoethanol were added and reacted at room temperature for 30 minutes. After the reaction, the product was filtered and dried to obtain 175.3 g (yield 95.7%) of the white powder Compound 1 having the following formula:

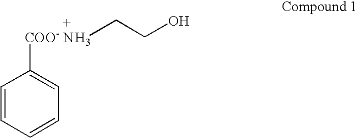

¹H NMR (400 MHz, DMSO-d6) δ in ppm: 2.8 (2H, CH₂—N), 3.6 (2H, CH—O), 7.3 to 7.8 (5H, Ph)

Melting point (DSC) : 146.5° C.

### Synthesis of Compound 2

Into 300 g of toluene, 122.12 g (1 mole) of benzoic acid and 75.11 g (1 mole) of 1-amino-2-propanol were added and reacted at room temperature for 30 minutes. After the reaction, the product was filtered and dried to obtain 195.1 g (yield 98.9%) of the white powder Compound 2 having the following formula:

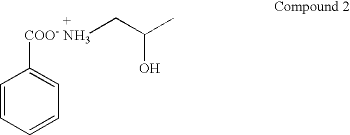

¹H NMR (400 MHz, DMSO-d6) δ in ppm: 1.1 (3H, CH3), 2.6, 2.8 (2H, CH₂—N), 3.8 (1H, CH—O), 7.3 to 7.8 (5H, Ph)

Melting point (DSC): 99.8° C.

### Synthesis of Compound 3

Into 600 g of toluene, 166.13 g (1 mole) of phthalic acid and 122.16 g (2 moles) of 2-aminoethanol were added and reacted at room temperature for 30 minutes. After the reaction, the toluene was removed in vacuo to obtain 270.0 g (yield 93.6%) of the liquid Compound 3 having the following formula:

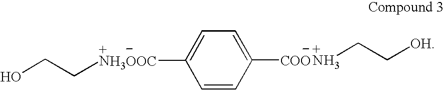

¹H NMR (400 MHz, DMSO-d6) δ in ppm: 2.7 (4H, CH₂—N), 3.5 (4H, CH—O), 7.5, 8.2 (4H, Ph)

### Synthesis of Compound 4

Into 300 g of toluene, 138.12 g (1 mole) of p-hydroxybenzoic acid and 61.08 g (1 mole) of 2-aminoethanol were added and reacted at room temperature for 30 minutes. After the reaction, the product was filtered and dried to obtain 196.5 g (yield 98.6%) of the white powder Compound 4 having the following formula:

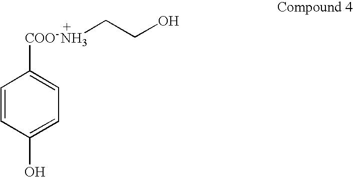

¹H NMR (400 MHz, DMSO-d6) δ in ppm: 2.8 (2H, CH₂—N), 3.6 (2H, CH—O), 6.7, 7.7 (4H, Ph)

Melting point (DSC): 174.8° C.

### Synthesis of Compound 5

Into 700 g of acetone, 200.3 g (1 mole) of lauric acid and 61.08 g (1 mole) of 2-aminoethanol were added and reacted at room temperature for 30 minutes. After the reaction, the product was filtered and dried to obtain 242.3 g (yield 92.7%) of the white powder Compound 5 having the following formula:

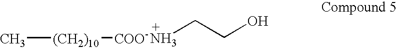

¹H NMR (400 MHz, DMSO-d6) δ in ppm: 0.8, 1.2, 1.4 (19H, CH₂, CH₃), 2.0 (2H, CH₂—COO), 2.6 (2H, CH₂—N), 3.4 (2H, CH—O)

Melting point (DSC): 65.3° C.

### Preparation of Samples

In each of the formulations shown in Table I, the ingredients other than the vulcanization accelerator and sulfur were kneaded in a 1.5 liter Banbury mixer for 5 minutes to obtain a master batch. This master batch was kneaded with the vulcanization accelerator and sulfur by an open roll to obtain a rubber composition.

TABLE I

| | Exp. No. | | | | | | | |
|---|---|---|---|---|---|---|---|---|
| | Comp. | | | | | | | Comp. |
| | Ex. 1 | Ex. 6 | Ex. 7 | Ex. 8 | Ex. 9 | Ex. 10 | Ex. 11 | Ex. 2 |
| Formulation (parts by mass) | | | | | | | | |
| NR*1 | 100 | 100 | 100 | 100 | 100 | 100 | 80 | 100 |
| SBR*2 | — | — | — | — | — | — | 20 | — |
| CB*3 | 50 | 50 | 50 | 50 | 50 | 50 | 50 | 50 |
| ZnO*4 | 3 | 3 | 3 | 3 | 3 | 3 | 3 | 3 |
| Stearic acid*5 | 1 | 1 | 1 | 1 | 1 | 1 | 1 | 1 |
| Antioxidant*6 | 1 | 1 | 1 | 1 | 1 | 1 | 1 | 1 |
| Sulfur*7 | 1.5 | 1.5 | 1.5 | 1.5 | 1.5 | 1.5 | 1.5 | 1.5 |
| Compound 1 | — | 0.5 | 1 | — | — | — | 1 | — |
| Compound 2 | — | — | — | 1 | — | — | — | — |
| Compound 3 | — | — | — | — | 1 | — | — | — |
| Compound 4 | — | — | — | — | — | 1 | — | — |
| Compound 5 | — | — | — | — | — | — | — | 1 |
| NS*8 | 1.2 | 1.2 | 1.2 | 1.2 | 1.2 | 1.2 | 1.2 | 1.2 |
| Evaluated properties | | | | | | | | |
| Rheometer | | | | | | | | |
| T90 (min) | 13.3 | 7.2 | 6.7 | 6.8 | 6.3 | 7.1 | 6.7 | 11.9 |
| Tensile test | | | | | | | | |
| M100 (MPa) | 2.6 | 2.9 | 3.0 | 2.9 | 2.8 | 2.8 | 2.9 | 2.7 |
| TB (MPa) | 30.1 | 30.1 | 29.9 | 31.2 | 31.4 | 30.9 | 30.7 | 29.6 |
| EB (%) | 500 | 495 | 487 | 505 | 511 | 503 | 492 | 505 |
| After aging (100° C. × 48 hr) | | | | | | | | |
| M100 (MPa) | 4.2 | 3.9 | 3.7 | 3.8 | 3.7 | 3.8 | 3.6 | 4.1 |
| TB (MPa) | 26.5 | 28.3 | 28.1 | 29.1 | 28.1 | 28.0 | 28.2 | 27.1 |
| EB (%) | 395 | 420 | 423 | 425 | 432 | 415 | 427 | 390 |

Footnote of Table I
*1RSS #3
*2Nipol 1712 made by Nippon Zeon
*3Diablack E made by Mitsubishi Chemical
*4Zinc Oxide Type 3 made by Seido Chemical Industry
*5Beads Stearic Acid YR made by NOF Corporation
*6Noccelar 6C made by Ouchi Shinko Chemical Industrial
*7Gold Flower sulfur powder made by Tsurumi Chemical
*8Noccelar NS-P made by Ouchi Shinko Chemical Industrial

### Test Method

### Rheometer

Based on ASTM D2084, the vulcanized properties of the rubber composition of the present invention at 150° C. were determined (ASTM method for cross-linked rubber properties using oscillating disk cure meter). T95 shows the time until the cross-linking density becomes 95%, that is, the time until vulcanization is substantially completed.

### Tensile Test

Each rubber composition obtained above was vulcanized at 150° C. for 30 minutes to prepare a 15 cm×15 cm×2 mm vulcanized sheet. From this vulcanized sheet, a JIS No. 3 dumbbell shaped test piece was punched out. According to JIS K6251, the modulus at 100% elongation (M100), the tensile strength at break (TB) and the elongation at break (EB) were found. Further, according to JIS K6257, the M100, TB and EB after aging at 100° C. for 48 hours were determined. The results are shown in Table I.

### INDUSTRIAL APPLICABILITY

The compounding agent for rubber vulcanization including an amino alcohol salt compound of an aromatic carboxylic acid (I) of the present invention has a high vulcanization acceleration effect to diene-based rubbers and the like. The vulcanized rubber obtained by vulcanizing an unvulcanized rubber composition containing a compounding agent for rubber vulcanization including an amino alcohol salt compound of an aromatic carboxylic acid (I) of the present invention shows a higher heat aging resistance than those obtained from an unvulcanized rubber composition containing a conventional vulcanization agent and/or a vulcanization accelerator.

1. A compounding agent for rubber vulcanization comprising an amino alcohol salt compound of an aromatic carboxylic acid having the formula (I) obtained by reacting an aromatic carboxylic acid with an amino alcohol: 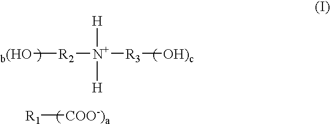
wherein R₁ is a C₆ to C₂₄ aromatic hydrocarbon which may have a hydroxy group and/or a substituent group, R₂ and R₃ are independently hydrogen or a C₁ to C₂₀ organic group which may have a hetero atom and/or a substituent group and R₂ and R₃ may bond with each other to form a ring, a is 1 or 2, b and c are 0 or 1 and b+c is 1 to 2.

2. A method for producing a compounding agent for rubber vulcanization comprising an amino alcohol salt compound of an aromatic carboxylic acid having the formula (I) comprising reacting an aromatic carboxylic acid having the formula (II) with an amino alcohol having the formula (III) by the following reaction: 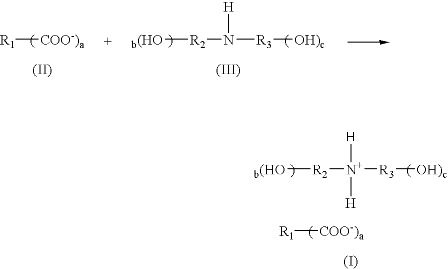
wherein R₁ is a C₆ to C₂₄ aromatic hydrocarbon which may have a hydroxy group and/or a substituent group, R₂ and R₃ are independently hydrogen or a C₁ to C₂₀ organic group which may have a hetero atom and/or a substituent group, and R₂ and R₃ may bond with each other to form a ring, a is 1 or 2, b and c are 0 or 1 and b+c is 1 to 2.

3. A method for producing a compounding agent for rubber vulcanization as claimed in claim 2, wherein the aromatic carboxylic acid is benzoic acid or phthalic acid.

4. A rubber composition comprising 30 parts by mass of at least one unvulcanized rubber component selected from the group consisting of diene-based rubbers and 0.05 to 10 parts by mass of a compounding agent for rubber vulcanization according to claim 1.
